(19) United States
(12) Patent Application Publication
(10) Pub. No.: US 2022/0064689 A1
(43) Pub. Date: Mar. 3, 2022

(54) AMPHOTERIC GLYCOLIPID BIOSURFACTANT AND ITS PREPARATION METHOD

(71) Applicant: Mikros Biochem, Costa Mesa, CA (US)

(72) Inventor: Wenjie Xia, Irvine, CA (US)

(73) Assignee: Mikros Biochem, Costa Mesa, CA (US)

(21) Appl. No.: 17/412,031

(22) Filed: Aug. 25, 2021

Related U.S. Application Data
(60) Provisional application No. 63/190,177, filed on May 18, 2021.

(30) Foreign Application Priority Data
Aug. 26, 2020 (CN) 112156719A

(51) Int. Cl. C12P 19/44; B01F 17/00; A01N 25/30; A01N 43/16

(52) U.S. Cl. CPC C12P 19/44; A01N 43/16; A01N 25/30; B01F 17/0042

(57) ABSTRACT

Provided herein are amphoteric glycolipid biosurfactants containing an amphoteric glycolipid biological surface active molecule preparation produced by acid precipitation of culture supernatant produced by sequentially culturing Pseudomonas, Candida, and Neurospora, for instance in a fermentation medium comprising a hydrophilic carbon source and a hydrophobic carbon source. Also described are amphoteric glycolipid biological surface active molecule preparations, and methods of making such preparations. The amphoteric molecules have anionic and cationic groups; example molecules include 17-L-[(2′-O-β-D-glucopyranosyl-β-D-Glucosyl)-O-]-octadecenoic acid amine-6′,6″ diacetate, and 17-L-[(2′-O-β-D-glucopyranosamine-β-D-rhamnosyl)-O-]-octadecenoic acid-6′,6″ diacetate. The amphoteric glycolipid biosurfactant has good compatibility with other types of surfactants, high temperature resistance and salt resistance, and is suitable for use in a variety of liquid systems.

### CROSS-REFERENCE TO RELATED APPLICATIONS

This application claims priority to and the benefit of the earlier filing date of China Patent Application No. CN112156719A, filed Aug. 26, 2020; and U.S. Provisional Application No. 63/190,177, filed on May 18, 2021. Each of these earlier-filed applications is incorporated by reference herein in its entirety.

### FIELD OF THE DISCLOSURE

The invention relates to amphoteric biosurfactants, preparation methods, and applications for using them. It also relates to an amphoteric glycolipid biosurfactant and its preparation method.

### BACKGROUND OF THE DISCLOSURE

Biosurfactants are a class of active substances with surface activity formed by microorganisms through their own metabolism. Such substances have hydrophilic and hydrophobic groups, and generally have a more complex structure than chemical surfactants. Therefore, usefulness of biosurfactants is very wide; biosurfactants have been widely used in industries such as chemical industry, medicine, cosmetics, late oil recovery, and environmental pollution control.

According to the structural characteristics of biosurfactants, they can be divided generally into polymers, lipopeptides, neutral lipids, phospholipids, etc. Biosurfactant-producing bacteria include Enterococcus species, Pseudomonas species, Streptomyces species, Bacillus licheniformis, Acinetobacter species, yeast, etc. Such microorganisms will secrete a mix of surfactant-containing metabolites in vitro during the growth process under specific culture condition(s), such as suitable carbon source, nitrogen source, organic nutrients, pH value, and temperature. This surfactant-containing metabolite mix is or contains a biological surface-active agent.

It is predicted that the cost to produce biosurfactants is only 30% of the cost of manufacturing synthetic surfactants. Since biosurfactants are non-toxic, from an ecological point of view, biosurfactants are more conducive to environmental protection than synthetic surfactants. Because biosurfactants have the above advantages and can be fermented and produced through biological metabolism and other means, they have received general attention from the bioengineering community. At the same time, biosurfactants have anti-tumor, anti-bacterial, anti-fungal, anti-viral, and anti-inflammatory activities. However, all biosurfactants that have been previously recognized are anionic or nonionic surfactants; there is no biosurfactant with cationic groups.

The types of chemically synthesized surfactants are very abundant, which makes the application fields of chemically synthesized surfactants relatively wide. In the practical application of conventional biosurfactants, some technical bottlenecks are often encountered that are caused by the absence of cationic groups. For example, in inhibiting bacteria and other biochemical activities, the effect of cations is more obvious than that of conventional surfactants; for example, problems such as high metal ion content in liquids and ultra-low pH can be overcome by adding cationic surfactants. Once different types of surfactants are introduced, under certain conditions, the overall effect will often be greatly reduced. Therefore, there continues to be a strong need for amphoteric biosurfactants and methods to prepare them, as amphoteric biosurfactant would have important application value, and would broaden the variety and function diversity of biosurfactants.

### SUMMARY OF THE DISCLOSURE

A first objective of embodiments of the present disclosure is to provide methods for preparing an amphoteric glycolipid with biosurface activity that can be made using a simple production process, at relatively low cost, and relying on widely available raw material source(s).

A second objective of embodiments of the present disclosure is to provide amphoteric glycolipid biosurfactants, including examples with excellent performance and multiple functions.

A third objective of embodiments of the present disclosure is to provide a synthesis method involving multi-bacteria, multi-stage combination, and fermentation in the same tank.

Embodiments of the present disclosure provide an amphoteric glycolipid biosurfactant, which includes as the main active component a fermentation broth of the amphoteric glycolipid biosurfactant molecules and contains the following chemical components: fatty acid, amphoteric glycolipid biosurfactant molecule, polysaccharide, sugar amines (that is, any amino sugar or mixture thereof), lipopeptides, ethanol, and fatty amines. In examples, this is an aqueous mix and the remaining volume is water (water not being generally considered an active component).

Examples of the herein provided amphoteric glycolipid biosurfactant have the following content ranges:

Fatty acid: 0.5-3.0%,

Polysaccharide: 0.1-1.0%,

Amphoteric glycolipid biosurface active molecules: 20.0-40.0%,

Glycosamine (which can be any amino sugar): 0.1%-0.5%,

Lipopeptide: 0.01-0.2%,

Ethanol: 1-3%,

Fatty amine: 0.1-0.5%.

Made up to 100% with water.

The polysaccharide contemplated in embodiments of the herein disclosed biosurfactants includes a polymerized polysaccharide polymer containing monosaccharides such as rhamnose, mannose, glucose, etc. The polymer biopolysaccharide in various embodiments includes one or more of xanthan gum, dextran, and gellan gum; the molecular weight is optionally controlled at 20-500,000 Daltons.

The fatty acids contemplated in embodiments of the herein disclosed biosurfactants include C₈-C₁₆ long-chain fatty acids, such as one or more of caprylic acid, capric acid, lauric acid, myristic acid and palmitic acid.

Glucosamine contemplated in embodiments of the herein disclosed biosurfactants includes glucosamine and its derivatives, such as glucosamine, N-acetylglycosamine, etc.; the molecular weight is less than 1000 Daltons.

The lipopeptides contemplated in embodiments of the herein disclosed biosurfactants include subtilisin, phenazines, iturin and other lipopeptides containing 7 amino acid chains with a molecular weight of 800-3000 Daltons. Subtilin is a closed-loop lipopeptide containing 7 amino acids as a hydrophilic group and a fatty acid chain with a chain length of 13-15. Iturin is a β-amino fatty acid (C₁₄-C₁₇) with seven α-amino acid residues, and a β-amino fatty acid to form a closed-loop lipopeptide. The ratio of subtilisin to phenazine in the lipopeptide can be 10:1-12:1, for instance.

The fatty amines contemplated in embodiments of the herein disclosed biosurfactants may include one or more of: lauryl amide, palmitic acid amide, stearamide, oleic acid amide, and stearyl oleamide.

The amphoteric glycolipid biosurfactant molecule contemplated in embodiments of the herein disclosed biosurfactant is obtained by mixing and sequential cultivation of strains such as Candida, Pseudomonas, Neurospora, etc., and then after separation, acid precipitation, filtration and concentration. The preparation process is exemplified as follows:

(1) Fermentation: Inoculate 5%-10% (v/v) Pseudomonas seed solution into prepared fermentation medium (see below) and cultivate/ferment for 24-36 hours. The temperature during the cultivation process can be within the range of 33-37° C. After 24-36 hours, add 5%-10% Candida seed solution into the fermented Pseudomonas culture solution, adjust the temperature to 23-26° C., and cultivate/ferment for 80-100 hours. After 80-100 hours, add 5%-10% Neurospora seed solution into the fermented Candida culture solution, adjust the temperature to 28-32° C., and cultivate/ferment for 48 hours. In embodiments, the seed solution is conventional LB medium, and the relevant bacterial inoculum in the seed culture is in the log (exponential) growth phase.

(2) Separation: The fermentation broth obtained in the above three stage fermentation process is sterilized by heating at 80-120° C. for 2 hours, then adjusted to pH=8.0-10.0, and subjected to three-phase high-speed centrifugation (*10000 g) to remove bacterial materials and residues (such as fats or oils), yielding a clear liquid middle layer that is harvested.

(3) Acid precipitation: The pH of the clear liquid in the middle layer is adjusted to pH=2-3, and the mixture is refrigerated it at 4-10° C. for 24 hours to precipitate components in the liquid.

(4) Filtration: The precipitate is collected through a ceramic membrane at 4-10° C. The precipitate is suspended/dissolved in an aqueous solution of pH>8; this preparation is then passed through a ceramic membrane to obtain an aqueous solution of amphoteric glycolipid bio-surface active molecules.

(5) Concentration: Optionally, the aqueous solution of amphoteric glycolipid bio-surface active molecules can be concentrated, for instance by 10-15 times, using vacuum concentration at 50° C.

An exemplary aqueous fermentation medium that is useful in the above three stage fermentation process includes: NaNO₃: 0.4-2.4%, FeCl₂: 0.002-0.006%, NaH₂PO₄: 0.25-1.5%, K₂HPO₄: 0.25-1.8%, MgSO₄.7H₂O: 0.005-0.015%, KCl: 0.05%-0.3%, Choline chloride: 0.05%-0.3%, Fatty amine: 0.5-1.0%, Corn syrup: 0.05%-0.3%, Hydrophobic carbon source: 3.0-5.0%, Hydrophilic carbon source: 0.1%-0.5% and yeast extract powder: 0.001-0.1%, adjusted to about pH 6-7.

After analysis of the aqueous solution of amphoteric glycolipid bio-surface active molecules prepared using the above methods, using HPLC-MS and other analytic methods, the composition was determined to include: structure identified as 17-L-[(2′-O-β-D-glucopyranosyl-β-D-glucosyl)-O-]-octadecenoic acid amine-6′,6″ Diacetate, and 17-L-[(2′-O-β-D-glucopyranosamine-β-D-rhamnosyl)-O-]-octadecenoic acid-6′,6″, a mixture of diacetates.

Provided herein are amphoteric glycolipid biosurfactants, an active component of which is a preparation of amphoteric glycolipid bio-surface active molecules made by culturing a plurality of different types of microorganisms as strains, for instance at 24-30° C., 100-200 rpm, and under aeration conditions, under shaking fermentation for 5-10 days. The resultant culture is then filtered, and desired fraction(s) are obtained by concentration and purification, which is based on the extract of the amphoteric glycolipid biological surface active fermentation broth as the main component, containing the following: C₈-C₁₄ chain length fatty acid 0.5-3.0%, amphoteric glycolipid biological surface active molecules 20.0-40.0%, polysaccharide 0.1-1.0%, fatty amine 0.1-0.5%, sugar amine 0.1%-0.5%, lipopeptide 0.01-0.2%, ethanol 1-3%, others are water. Among them, the amphoteric glycolipid biosurfactant molecule is the main functional component, and its structure has both anionic and cationic groups, and in one example it is 17-L-[(2′-O-β-D-glucopyranosyl-β-D-Glucosyl)-O-]-octadecenoic acid amine-6′,6″ diacetate, or in another example it is 17-L-[(2′-O-β-D-glucopyranosamine-β-D-rhamnosyl)-O-]-octadecenoic acid-6′,6″ diacetate. Embodiments of the amphoteric glycolipid biosurfactant has good compatibility with other types of surfactants, high temperature resistance and salt resistance, and is suitable for a variety of liquid systems.

### BRIEF DESCRIPTION OF THE DRAWINGS

FIGS. 1A, 1B show the structures of 17-L-[(2′-O-β-D-glucopyranosyl-β-D-glucosyl)-O-]-octadecenoic acid amine-6′,6″ diacetate (FIG. 1A); and FIG. 1B is 17-L-[(2′-O-β-D-glucopyranosylamino-β-D-rhamnosyl)-O-]-octadecyl Enoic acid-6′,6″ diacetate.

FIG. 2 is a graph illustrating interfacial tension with kerosene.

FIG. 3 illustrates compatibility with calcium and magnesium ions based on turbidity of the solution.

FIGS. 4A, 4B show the structures of 17-L-[(2′-O-β-D-glucopyranosylamino-β-D-rhamnosyl)-O-]-18 Enoic acid-6′, 6″ diacetate (FIG. 4A), and 17-L-[(2′-O-β-D-glucopyranosyl-β-D-glucosyl)-O-]-octadecene Acid amine-6′, 6″ diacetate (FIG. 4B).

FIG. 5 is a graph illustrating interfacial tension with kerosene.

FIG. 6. Illustrates compatibility with calcium and magnesium ions based on turbidity of the solution.

The Exemplary Embodiments and Examples below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### EXEMPLARY EMBODIMENTS

1. An amphoteric glycolipid biosurfactant including: an amphoteric glycolipid biological surface active molecule preparation produced by acid precipitation of fermentation culture supernatant produced by sequentially culturing Pseudomonas aeruginosa, Candida albicans, and Neurospora crassa in a fermentation medium including at least one hydrophilic carbon source and at least one hydrophobic carbon source; one or more fatty acids; one or more polysaccharides; one or more sugar amines; one or more lipopeptides; ethanol; one or more fatty amines; and water, wherein the amphoteric glycolipid biological surface active molecule preparation is the predominant active component.
2. The amphoteric glycolipid biosurfactant of embodiment 1, including: 0.5-3.0% fatty acids, 0.1-1.0% polysaccharide: 20.0-40.0% amphoteric glycolipid biological surface active molecule preparation, 0.1%-0.5% glycosamine, 0.01-0.2% lipopeptide, 1-3% ethanol, and 0.1-0.5% fatty amine.
3. The amphoteric glycolipid biosurfactant of embodiment 1, wherein the polysaccharide: includes a polysaccharide polymer including one or more of rhamnose, mannose, or glucose; and has a molecular weight greater than 200,000 Daltons.
4. The amphoteric glycolipid biosurfactant of embodiment 1, wherein the fatty acid includes a C₈-C₁₆ long-chain fatty acid.
5. The amphoteric glycolipid biosurfactant of embodiment 1, wherein the sugar amine includes N-acetylglycosamine of glucose with a molecular weight of less than 1000 Daltons. 6. The amphoteric glycolipid biosurfactant of embodiment 1, wherein the lipopeptides include at least one lipopeptide containing 7-14 amino acid chains, with a molecular weight of 800-3000 Daltons.
7. The amphoteric glycolipid biosurfactant of embodiment 6, wherein the lipopeptides include one or more of subtilisin, a phenazine, and/or iturin.
8. The amphoteric glycolipid biosurfactant of embodiment 7, wherein the lipopeptide includes subtilisin and phenazine in a ratio of subtilisin to phenazine of 10:1-12:1.
9. The amphoteric glycolipid biosurfactant of embodiment 1, wherein the structure of at least one amphoteric glycolipid biological surface active molecule in the preparation includes fatty amine or Glycosamine or both; and the molecule has a weight of less than 1400 Daltons.
10. The amphoteric glycolipid biosurfactant of embodiment 1, wherein amphoteric glycolipid biological surface active molecule preparation includes one or more of:
17-L-[(2′-O-β-D-glucopyranosyl-β-D-glucosyl)-O-]-octadecenoic acid amine-6′,6″ diacetate;
17-L-[(2′-O-β-D-glucopyranosamine-β-D-rhamnosyl)-O-]-octadecenoic acid-6′,6″, diacetate;
17-L-[(2′-O-β-D-glucopyranosylamino-β-D-rhamnosyl)-O-]-octadecylenoic acid-6′,6″ diacetate;
17-L-[(2′-O-β-D-glucopyranosylamino-β-D-rhamnosyl)-O-]-18 Enoic acid-6′,6″ diacetate; or
17-L-[(2′-O-β-D-glucopyranosyl-β-D-glucosyl)-O-]-octadecene acid amine-6′,6″ diacetate.
11. The amphoteric glycolipid biosurfactant of embodiment 1, wherein the fatty amine includes one or more of lauryl amide, palmitic acid amide, stearamide, oleic acid amide, or stearyl oleamide.
12. An amphoteric glycolipid biological surface active molecule preparation produced by acid precipitation of fermentation culture supernatant produced by sequentially culturing Pseudomonas aeruginosa, Candida albicans, and Neurospora crassa in a fermentation medium.
13 The amphoteric glycolipid biological surface active molecule preparation of embodiment 12, which is obtained by sequential cultivation of Candida, Pseudomonas, and Neurospora in the fermentation medium to produce a fermentation broth mixture, separation of fermentation culture supernatant from the fermentation broth mixture, acid precipitation of material from the fermentation culture supernatant to produce a precipitate, and filtration of the precipitate.
14. The amphoteric glycolipid biological surface active molecule preparation of embodiment 12, wherein one or more of: the Candida are cultured at 33-37° C.; the Pseudomonas are cultured at 23-26° C.; the Neurospora are cultured at 28-32° C.; the fermentation medium includes at least one hydrophilic carbon source and at least one hydrophobic carbon source; the Candida are cultured for 24-36 hours; the Pseudomonas are cultured for 80-100 hours; and/or the Neurospora are cultured for 36-48 hours.
15. The amphoteric glycolipid biological surface active molecule preparation of embodiment 14, wherein the at least one hydrophilic carbon source includes sucrose, glucose, and/or molasses; and or wherein the at least one hydrophobic carbon source includes vegetable oil, animal oil, and/or a petroleum hydrocarbon compound.
16. The amphoteric glycolipid biological surface active molecule preparation of embodiment 12, production of which further includes an extraction process including: (1) separation, including lowering the fermentation broth to pH=8.0-10.0, centrifuging the pH adjusted broth at *10000 g to remove bacterial debris and residual oil, and harvesting a clear middle liquid layer; (2) acid precipitation, including adjusting the middle clear liquid layer to pH=2-3, and refrigerating at 4-10° C. for 24 hours to produce a precipitate; (3) filtration, including collecting the precipitate through a ceramic membrane filter at 4-10° C., dissolving/suspending the precipitate in an aqueous solution of pH>8, and filtering the suspended solution through a ceramic membrane filter to obtain an aqueous preparation of amphoteric glycolipid biosurface active molecule; and, optionally (4) concentrating preparation in vacuo at 50° C.
17. The amphoteric glycolipid biological surface active molecule preparation of embodiment 12, wherein the Pseudomonas is cultured at 33-37° C. for 24-36 hours, and then connected to Candida for 80-100 hours at 23-26° C. Finally, insert the Neurospora bacteria and cultivate at 28-32° C. for 36-48 hours.
18. The amphoteric glycolipid biological surface active molecule preparation of embodiment 12, wherein the fermentation medium is at pH 6-7 and includes: NaNO₃: 0.4-2.4%, FeCl₂: 0.002-0.006%, NaH₂PO₄: 0.25-1.5%, K₂HPO₄: 0.25-1.8%, MgSO₄.7H₂O: 0.005-0.015%, KCl: 0.05%-0.3%, choline chloride: 0.05%-0.3%, fatty amine: 0.5-1.0%, corn syrup: 0.05%-0.3%, hydrophobic carbon source: 3-5%, hydrophilic carbon source: 0.1%-0.5%, yeast extract powder: 0.001-0.1%, and trace elements: Zn, Mn, Ca.
19. The amphoteric glycolipid biological surface active molecule preparation of embodiment 12, which includes at least one molecule having the structure:
17-L-[(2′-O-β-D-glucopyranosyl-β-D-glucosyl)-O-]-octadecenoic acid amine-6′,6″ diacetate;
17-L-[(2′-O-β-D-glucopyranosamine-β-D-rhamnosyl)-O-]-octadecenoic acid-6′,6″, diacetate;
17-L-[(2′-O-β-D-glucopyranosylamino-β-D-rhamnosyl)-O-]-octadecylenoic acid-6′,6″ diacetate;
17-L-[(2′-O-β-D-glucopyranosylamino-β-D-rhamnosyl)-O-]-18 Enoic acid-6′, 6″ diacetate; or
17-L-[(2′-O-β-D-glucopyranosyl-β-D-glucosyl)-O-]-octadecene acid amine-6′, 6″ diacetate.
20. The amphoteric glycolipid biological surface active molecule preparation of embodiment 12, which exhibits improved surface activity, improved bacteriostasis activity, improved emulsification activity, or a combination of two or more thereof.
21. A method of making an amphoteric glycolipid biological surface active molecule preparation, the method including: acid precipitation of fermentation culture supernatant produced by sequentially culturing Pseudomonas aeruginosa, Candida albicans, and Neurospora crassa in a fermentation medium.
22. The method of embodiment 21, including sequential cultivation of Candida, Pseudomonas, and Neurospora in the fermentation medium to produce a fermentation broth mixture, separation of fermentation culture supernatant from the fermentation broth mixture, acid precipitation of material from the fermentation culture supernatant to produce a precipitate, and filtration of the precipitate.
23. The method of embodiment 21, wherein one or more of: the Candida are cultured at 33-37° C.; the Pseudomonas are cultured at 23-26° C.; the Neurospora are cultured at 28-32° C.; the fermentation medium includes at least one hydrophilic carbon source and at least one hydrophobic carbon source; the Candida are cultured for 24-36 hours; the Pseudomonas are cultured for 80-100 hours; and/or the Neurospora are cultured for 36-48 hours.
24. The method of embodiment 23, wherein the at least one hydrophilic carbon source includes sucrose, glucose, and/or molasses; and or wherein the at least one hydrophobic carbon source includes vegetable oil, animal oil, and/or a petroleum hydrocarbon compound.
25. The method of embodiment 21, production of which further includes an extraction process including: (1) separation, including lowering the fermentation broth to pH=8.0-10.0, centrifuging the pH adjusted broth at *10000 g to remove bacterial debris and residual oil, and harvesting a clear middle liquid layer; (2) acid precipitation, including adjusting the middle clear liquid layer to pH=2-3, and refrigerating at 4-10° C. for 24 hours to produce a precipitate; (3) filtration, including collecting the precipitate through a ceramic membrane filter at 4-10° C., dissolving/suspending the precipitate in an aqueous solution of pH>8, and filtering the suspended solution through a ceramic membrane filter to obtain an aqueous preparation of amphoteric glycolipid biosurface active molecule; and, optionally (4) concentrating preparation in vacuo at 50° C.
26. The method of embodiment 21, wherein the Pseudomonas is cultured at 33-37° C. for 24-36 hours, and then connected to Candida for 80-100 hours at 23-26° C. Finally, insert the Neurospora bacteria and cultivate at 28-32° C. for 36-48 hours.
27. The method of embodiment 21, wherein the fermentation medium is at pH 6-7 and includes: NaNO₃: 0.4-2.4%, FeCl₂: 0.002-0.006%, NaH₂PO₄: 0.25-1.5%, K₂HPO₄: 0.25-1.8%, MgSO₄.7H₂O: 0.005-0.015%, KCl: 0.05%-0.3%, choline chloride: 0.05%-0.3%, fatty amine:
0.5-1.0%, corn syrup: 0.05%-0.3%, hydrophobic carbon source: 3-5%, hydrophilic carbon source: 0.1%-0.5%, yeast extract powder: 0.001-0.1%, and trace elements: Zn, Mn, Ca.
28. The method of embodiment 17, which includes at least one molecule having the structure:
17-L-[(2′-O-β-D-glucopyranosyl-β-D-glucosyl)-O-]-octadecenoic acid amine-6′,6″ diacetate;
17-L-[(2′-O-β-D-glucopyranosamine-β-D-rhamnosyl)-O-]-octadecenoic acid-6′,6″, diacetate;
17-L-[(2′-O-β-D-glucopyranosylamino-β-D-rhamnosyl)-O-]-octadecylenoic acid-6′,6″ diacetate;
17-L-[(2′-O-β-D-glucopyranosylamino-β-D-rhamnosyl)-O-]-18 Enoic acid-6′, 6″ diacetate; or
17-L-[(2′-O-β-D-glucopyranosyl-β-D-glucosyl)-O-]-octadecene acid amine-6′, 6″ diacetate.

### Example 1. Preparation of Amphoteric Glycolipid Biosurfactant (1)

1. Bacteria: Amphoteric biological surface active molecules were produced using three bacteria fermented in sequence in the same medium. These bacteria are exemplified by: Pseudomonas (ATCC 15442™; Pseudomonas aeruginosa (Schroeter) Migula; U.S. Pat. No. 5,719,113), Candida (ATCC 10231™; Candida albicans (Robin) Berkhout; Lingappa et al., Science 163(3863):192-194, 1969), and Neurospora (ATCC 18889™; Neurospora crassa Shear et Dodge; Maligie & Seliternnikoff, Antimicrob Agents Chemother. 49(7):2851-2856, 2005). Exemplary growing conditions for these bacteria are known, and can be found, for instance through the American Type Culture Collection data for each strain.

2. Medium: NaNO₃: 0.4%, FeCl₂: 0.002%, NaH₂PO₄: 0.25%, K₂HPO₄: 0.25%, MgSO₄.7H₂O: 0.005%, KCl: 0.05%, Choline chloride: 0.05%, fatty amine: 0.5, corn syrup: 0.05%, soybean oil and lauramide (1:1); 3.0%, glucose: 0.1%; yeast extract powder: 0.001%, pH 6-7.

3. Seed shake flask culture: The slant strains were cultivated for 36-48 hours, and the 3 single slants were transferred to a 1 liter Erlenmeyer flask. The Erlenmeyer flask was filled with 200 ml of LB medium. Pseudomonas was cultivated at about 35° C.; Candida was cultivated at around 25° C.; and Neurospora was cultivated at 30° C.; each were cultivated for 36 hours to form seed cultures.

4. 20 L Fermenter Amphoteric Glycolipid Production

(1) Fermentation: 5% Pseudomonas inoculum was used, by adding Pseudomonas seed solution (500 ml) into the prepared 10 L of medium (see (2) above); this was cultivated at 35° C. for 36 hours, at pH 6.5 (adjusted with 1 N HCl and NaOH), 0.4 ppv ventilation, 200 rpm stirring speed. 36 hours later, 6% Candida seed solution (600 ml) was added into the Pseudomonas-fermented culture solution, the temperature was adjusted to 23-26° C., and the second stage was incubated for 80-100 hours. After 100 hours, 7% (v/v) Neurospora seed solution was added to the Pseudomonas and Candida fermented culture solution, the temperature was adjusted to 29° C., and the third stage was cultured/cultured for 48 hours.

(2) Separation: The fermentation broth obtained from the above three stage fermentation process was sterilized at 100° C. for 2 hours, adjusted to pH=8.0, and then subjected to three-phase high-speed centrifugation (*10000 g) to remove bacterial cells and residual oil to obtain a clear middle layer.

(3) Acid precipitation: the pH of the clear liquid in the middle layer to was adjusted to pH=2, and the mixture was precipitated by refrigerating at 4° C. for 24 hours.

(4) Filtration: The resultant precipitate was filtered through a ceramic membrane (0.01 mm) at 4° C., and the precipitate was suspended/dissolved with an aqueous solution of pH=8. That solution was then passed through a ceramic membrane (0.01 mm) to obtain an aqueous solution of amphoteric glycolipid biological surface active molecules.

(5) Concentration: The primary aqueous solution of amphoteric glycolipid biological surface active molecules was concentrated by 10 fold, using vacuum concentration at 50° C.

TABLE 1
Biological surface activity fermentation results of amphoteric glycolipids in 20 L fermentor.

| | Fermentation | | | Stirring | Ventilation | Glycolipid | Substrate | Electrical |
|---|---|---|---|---|---|---|---|---|
| Stage | Time | Strain | pH | rpm | vvm | (g/L) | Yield % | Property |
| 1 | 36 | Pseudomonas | 7.0-7.5 | 200 | 0.4 | 45.68 | 60.78 | −/o |
| 2 | 80 | Candida | 6.0-7.0 | 200 | 0.4 | 50.37 | 65.24 | −/o/+ |
| 3 | 48 | Neurospora | 5.5-6.0 | 200 | 0.3 | 52.52 | 70.19 | −/+ |

Key: − represents anion, o represents non-ion, + represents cation; the content values are different in different batches.

The content of glycolipid fermentation liquid was determined by orcinol analysis (see, e.g., Laabei et al., Appl Microbiol Biotech. 98(16):7199-7209, 2014); other analyses may be used (e.g., see Izumi & Sawada, Lipids 36(1):97-101, 2001).

5. Chemical Structure Analysis of Biosurfactant Molecules of Amphoteric Glycolipids.

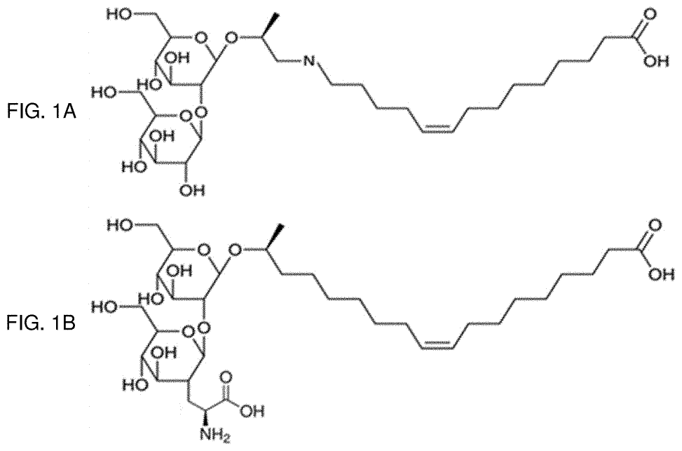

Using high performance liquid chromatography mass spectrometry, Zeta potential and electrophoresis experiments the structure (FIG. 1) and charge type of glycolipid was determined (see Table 1). The structure of amphoteric glycolipid type surface-active molecule is similar to that shown in FIGS. 1A, 1B, where N atoms exist in the structure in the form of secondary amines or sugar amines, which are present in both monosaccharide molecules and fatty chain structures; at the same time, the monosaccharide part is important. It is a double bond; the aliphatic chain part can be single-chain with double bonds, and contains between 18 carbon atoms.

FIGS. 1A, 1B. The most similar structures identified for the amphoteric glycolipid surfactant molecule are 17-L-[(2′-O-β-D-glucopyranosyl-β-D-glucosyl)-O-]-octadecenoic acid amine-6′,6″ diacetate (FIG. 1A); and FIG. 1B is 17-L-[(2′-O-β-D-glucopyranosylamino-β-D-rhamnosyl)-O-]-octadecyl Enoic acid-6′,6″ diacetate.

The above-produced amphoteric glycolipid biosurfactant molecules were integrated into an exemplary amphoteric biosurfactant composition as follows:

Fatty acid: 0.5%,

Polysaccharide: 0.1%,

Amphoteric glycolipid biosurface active molecules: 20.0%,

Glycosamine: 0.1%,

Lipopeptide: 0.01%,

Ethanol: 1%,

Fatty amine: 0.1%.

The remaining volume was made up with water.

6. Interfacial Activity of Amphoteric Glycolipid Biosurface Active Molecules.

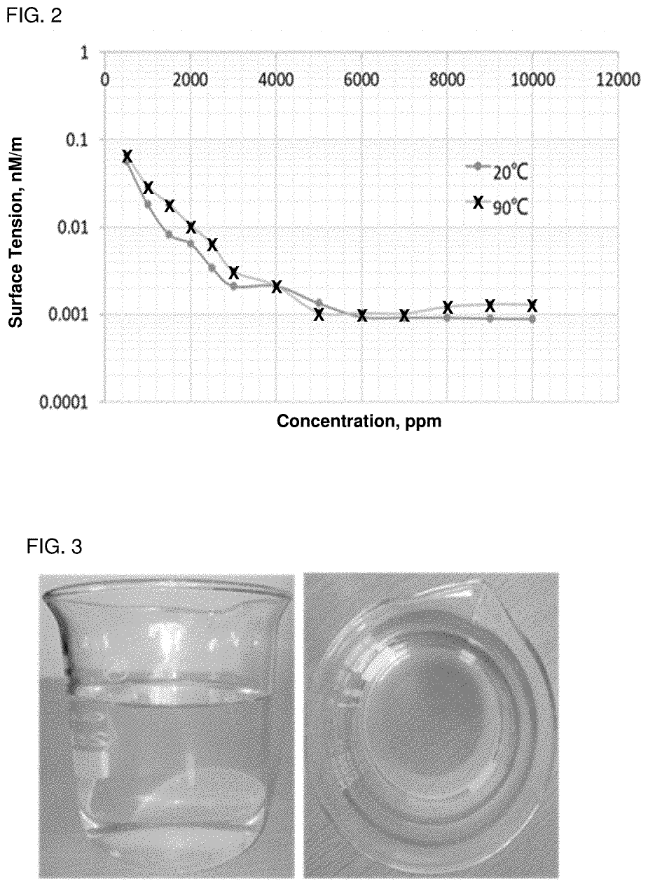

The exemplary amphoteric surfactant prepared above was diluted into solutions of different concentrations with pure water, and the interfacial tension between the solutions of different concentrations and kerosene was tested using the spin-drop method. The temperatures tested were 20° C. and 90° C. FIG. 2 is a graph illustrating the interfacial tension with kerosene.

FIG. 2 shows the results with the new biological amphoteric surfactant. The minimum interfacial tension between the surfactant and kerosene is 0.0008-0.001 mN/m, and both can reach ultra-low interfacial tension. The interfacial tension of other surfactant solutions and kerosene reported in the literature is greater than 0.01 nM/m, so the herein described amphoteric biosurfactant molecule has higher surface interfacial activity.

7. Compatibility with Metal Cations.

The amphoteric surfactant prepared as described above was diluted with pure water to make a 10,000 ppm aqueous solution, and then calcium chloride and magnesium chloride were added separately to a concentration of 20,000 ppm. FIG. 3 illustrates compatibility with calcium and magnesium ions based on turbidity of the solution; calcium chloride was added to the beaker on the left, and magnesium chloride to the beaker on the right.

It can be seen from FIG. 3 that, in the presence of calcium and magnesium ions up to 20,000 ppm, the solution remains clear, indicating that the new amphoteric biological surface active molecule preparation (and the amphoteric surfactant prepared using it) is compatible with cationic calcium and magnesium ions. Compared with the literature, general biosurfactant molecules cannot tolerate calcium and magnesium ions exceeding 3000 ppm.

8. Antibacterial Activity Analysis

Using the protocol set out in the national standard test WS/T 650-2019 “Evaluation Method of Antibacterial and Antibacterial Effects”, the amphiphilic biosurfactant produced as described above was analysed for antibacterial effects. Compared with the conventional bacteriostatic agent sodium benzoate, the provided amphoteric biosurfactants have higher antibacterial effects on bacteria (e.g., Staphylococcus aureus) and fungi (e.g., Candida albicans).

TABLE 2
Antibacterial analysis of amphoteric biosurfactants.

| | Amphoteric Surfactant | | | | | |
|---|---|---|---|---|---|---|
| Bacteria | Concentration | Day 0 | Day 7 | Day 14 | Day 21 | Day 28 |
| CFU/mL | 0.1% aqueous solution | 3.8 × 10⁶ | 8.8 × 10 | <10 | <10 | <10 |
| Staphylococcus | 0.3% aqueous solution | 3.3 × 10⁶ | 2.1 × 10 | <10 | <10 | <10 |
| aureus | 0.5% aqueous solution | 4.8 × 10⁶ | 1.8 × 10 | <10 | <10 | <10 |
| (ATCC 6538) | 1.0% aqueous solution | 4.3 × 10⁶ | 2.9 × 10 | <10 | <10 | <10 |
| | 1.5% aqueous solution | 4.9 × 10⁶ | 2.5 × 10 | <10 | <10 | <10 |
| | 2.0% aqueous solution | 3.2 × 10⁶ | 2.3 × 10 | <10 | <10 | <10 |
| | 4.0% aqueous solution | 4.8 × 10⁶ | 8.4 × 10 | <10 | <10 | <10 |
| | 4.0% sodium benzoate | 5.0 × 10⁶ | 4.9 × 10² | 8.7 × 10¹ | <10 | <10 |
| | (comparative) | | | | | |
| CFU/mL | 0.1% aqueous solution | 4.7 × 10⁵ | 8.9 × 10 | <10 | <10 | <10 |
| Candida | 0.3% aqueous solution | 4.4 × 10⁵ | 8.1 × 10 | <10 | <10 | <10 |
| albicans | 0.5% aqueous solution | 4.6 × 10⁵ | 8.0 × 10 | <10 | <10 | <10 |
| (ATCC 10231) | 1.0% aqueous solution | 3.3 × 10⁵ | 8.7 × 10 | <10 | <10 | <10 |
| | 1.5% aqueous solution | 3.7 × 10⁵ | 6.9 × 10 | <10 | <10 | <10 |
| | 2.0% aqueous solution | 3.8 × 10⁵ | 5.8 × 10 | <10 | <10 | <10 |
| | 4.0% aqueous solution | 4.9 × 10⁵ | 1.2 × 10 | <10 | <10 | <10 |
| | 4.0% sodium benzoate | 4.0 × 10⁵ | 3.4 × 10² | 3.0 × 10¹ | <10 | <10 |
| | (comparative) | | | | | |

### Example 2: Preparation of Amphoteric Glycolipid Biosurfactant (2)

1. Bacteria: Amphoteric biological surface active molecules were produced by cultivated with three bacteria Pseudomonas (ATCC 27853), Candida (ATCC 750) and Neurospora (ATCC 36935) in sequence.

2. Medium: NaNO₃: 1.4%, FeCl₂: 0.005%, NaH₂PO₄: 0.4%, K₂HPO₄: 0.4%, MgSO₄.7H₂O: 0.01%, KCl: 0.09%, Choline chloride: 0.05%, fatty amine: 0.5, corn syrup: 0.1%, soybean oil and lauramide (ratio 1:1): 4.0%, glucose: 0.3%; yeast extract powder: 0.004%, pH 6-7.

3. Seed shake flask culture: The slant strains were cultivated for 36-48 hours, and the 3 single slants were transferred to a 1 liter Erlenmeyer flask. The Erlenmeyer flask was filled with 200 ml of LB medium. Cultured at around 25° C., and Neurospora at around 30° C. for 30 hours.

4. 20 L Fermenter Amphoteric Glycolipid Production

(1) Fermentation: 8% Pseudomonas seed solution (800 ml) was inoculated into the prepared 10 L culture medium and fermented at 35° C. for 36 hours, at pH 6.5 (adjusted with 1 N HCl and NaOH), 0.5 ppv ventilation, 200 rpm stirring speed. 36 hours later, 9% Candida seed solution (900 ml) was added to the above culture solution, the temperature adjusted to 23-26° C., and the mixture incubated for 90 hours. After 90 hours, 10% of the Neurospora seed solution (1000 ml) was added to the above-mentioned culture solution, the temperature was adjusted to 30° C., and the mixture was cultured for 48 hours.

(2) Separation: The fermentation broth obtained from the above three stage fermentation process was sterilized at 100° C. for 2 hours, adjusted to pH=8.0, and then subjected to three-phase high-speed centrifugation (*10000 g) to remove bacterial cells and residual oil to obtain a clear middle layer.

(3) Acid precipitation: the pH of the clear liquid in the middle layer was adjusted to 2, and then it was refrigerated at 4° C. for 24 hours.

(4) Filtration: The resultant precipitate was collected by filtration through a ceramic membrane (0.01 mm) at 4° C., and then the precipitate was dissolved/suspended in an aqueous solution of pH=8. It was then passed through a second ceramic membrane (0.01 mm) to obtain the desired aqueous solution of amphoteric glycolipid biological surface active molecules.

(5) Concentration: The aqueous solution of amphoteric glycolipid biological surface active molecules was concentrated by 10 times using vacuum concentration at 50° C.

TABLE 3
Biological surface activity fermentation results of amphoteric glycolipids in a 20 L fermentor.

| | Fermentation | | | Stirring | Ventilation | Glycolipid | Substrate | Electrical |
|---|---|---|---|---|---|---|---|---|
| Stage | Time | Strain | pH | rpm | vvm | (g/L) | Yield % | Property |
| 1 | 36 | Pseudomonas | 7.0-7.5 | 200 | 0.4 | 50.21 | 64.12 | −/o |
| 2 | 90 | Candida | 3.5-4.5 | 200 | 0.4 | 53.32 | 66.34 | −/o/+ |
| 3 | 48 | Neurospora | 5.5-6.0 | 200 | 0.3 | 55.52 | 70.43 | −/+ |

Key: − represents anion, o represents non-ion, + represents cation; the above content values are different in different batches.

The content of the glycolipid fermentation liquid was determined by orcinol analysis.

5. Chemical Structure Analysis of Biosurfactant Molecules of Amphoteric Glycolipids.

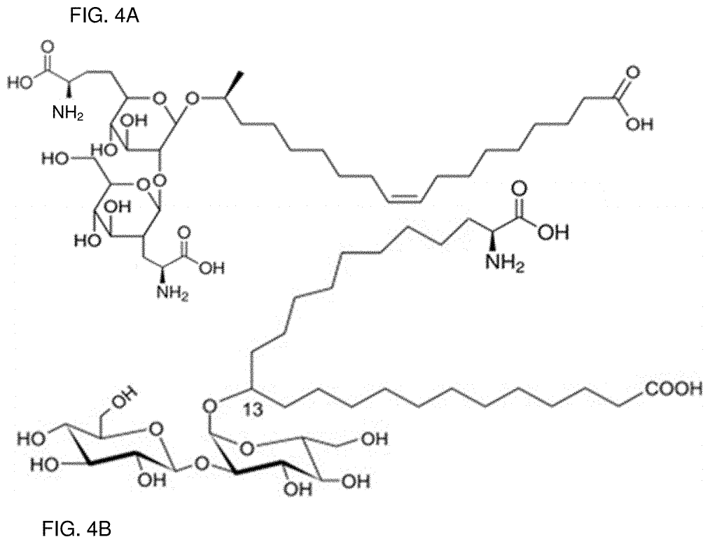

High performance liquid chromatography, mass spectrometry, Zeta potential, and electrophoresis experiments were used to determine the structure (FIGS. 4A, 4B) and charge type of glycolipid produced (see Table 3). The structure of amphoteric glycolipid type surface-active molecule is similar to those shown in FIGS. 4A, 4B, where N atoms exist in the structure in the form of secondary amines or sugar amines, which are present in both monosaccharide molecules and fatty chain structures; at the same time, the monosaccharide part is important it is a double pond; the aliphatic chain part can be single chain with double bonds, and contains between 18 carbon atoms, as shown.

FIGS. 4A, 4B. The similar structures identified for the amphoteric glycolipid surfactant molecule is 17-L-[(2′-O-β-D-glucopyranosylamino-β-D-rhamnosyl)-O-]-18 Enoic acid-6′, 6″ diacetate (FIG. 4A), and 17-L-[(2′-O-β-D-glucopyranosyl-β-D-glucosyl)-O-]-octadecene Acid amine-6′, 6″ diacetate (FIG. 4B).

The above-produced amphoteric glycolipid biosurfactant molecules were integrated into an exemplary amphoteric biosurfactant composition as follows:

Fatty acid: 3.0%,

Polysaccharide: 1.0%,

Amphoteric glycolipid biosurface active molecules: 40.0%,

Glycosamine: 0.5%,

Lipopeptide: 0.2%,

Ethanol: 3.0%,

Fatty amine: 0.5%.

The remaining volume was made up with water.

6. Interfacial Activity of Amphoteric Glycolipid Biosurface Active Molecules.

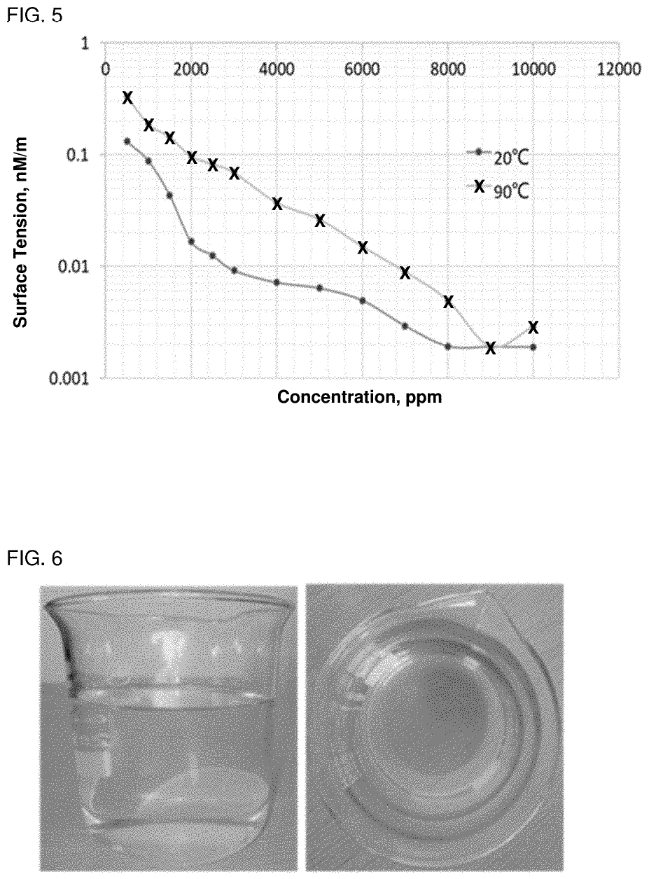

The exemplary amphoteric surfactant prepared above was diluted into solutions of different concentrations with pure water, and the interfacial tension between the solutions of different concentrations and kerosene was tested by the spinning drop method. The temperatures tested were 20° C. and 90° C. FIG. 5 is a graph illustrating the interfacial tension with kerosene.

It can be seen from the results (FIG. 5) that this new type of structure of amphoteric glycolipid biosurface active molecules (and the biosurfactant prepared using that mixture) is slightly different from that illustrated in Example 1, but the lowest interfacial tension with kerosene is 0.001-0.003 mN/m, which can also achieve ultra-low interfacial tension The interfacial tension of other surfactant solutions and kerosene reported in the literature is greater than 0.01 nM/m, so the herein described amphoteric biosurfactant molecule has higher surface interfacial activity.

7. Compatibility with Metal Cations.

The amphoteric surfactant prepared above was prepared with pure water to make a 10,000 ppm aqueous solution, and then calcium chloride and magnesium chloride were added separately to a concentration of 20,000 ppm FIG. 6. Illustrates compatibility with calcium and magnesium ions; calcium chloride was added to the beaker on the left, and magnesium chloride to the beaker on the right. Observe the turbidity of the solution.

It can be seen from FIG. 6 that, in the presence of calcium and magnesium ions up to 20,000 ppm, the solution remains clear, indicating that the new amphoteric biological surface active molecule preparation (and the amphoteric surfactant prepared using it) described in this Example is compatible with cationic calcium and magnesium ions. Compared with the literature, general biosurfactant molecules cannot tolerate calcium and magnesium ions exceeding 3000 ppm.

8. Antibacterial Activity Analysis

Using the protocol set out in the national standard test WS/T 650-2019 “Evaluation Method of Antibacterial and Antibacterial Effects”, the amphiphilic biosurfactant produced as described above was analysed for antibacterial effects. Compared with the conventional bacteriostatic agent sodium benzoate and the amphoteric biosurfactant of Example I, the amphoteric biosurfactant of this Example has higher antibacterial effects on bacteria (e.g., Staphylococcus aureus) and fungi (e.g., Candida albicans).

TABLE 4
Antibacterial experiment of amphoteric biosurfactants.

| | Amphoteric Surfactant | | | | | |
|---|---|---|---|---|---|---|
| Bacteria | Concentration | Day 0 | Day 7 | Day 14 | Day 21 | Day 28 |
| CFU/mL | 0.1% aqueous solution | 4.8 × 10⁶ | 2.8 × 10 | <10 | <5 | <3 |
| Staphylococcus | 0.3% aqueous solution | 4.3 × 10⁶ | 3.1 × 10 | <10 | <10 | <3 |
| aureus | 0.5% aqueous solution | 3.8 × 10⁶ | 1.8 × 10 | <10 | <10 | <3 |
| (ATCC 6538) | 1.0% aqueous solution | 4.3 × 10⁶ | 1.9 × 10 | <10 | <10 | <3 |
| | 1.5% aqueous solution | 3.9 × 10⁶ | 1.5 × 10 | <10 | <10 | <3 |
| | 2.0% aqueous solution | 4.2 × 10⁶ | 1.3 × 10 | <10 | <10 | <3 |
| | 4.0% aqueous solution | 4.8 × 10⁶ | 2.4 × 10 | <10 | <10 | <3 |
| | 4.0% sodium benzoate | 5.2 × 10⁶ | 3.9 × 10² | 8.7 × 10¹ | <10 | <3 |
| | (comparative) | | | | | |
| CFU/mL | 0.1% aqueous solution | 4.7 × 10⁵ | 6.9 × 10 | <10 | <10 | <3 |
| Candida albicans | 0.3% aqueous solution | 5.4 × 10⁵ | 7.1 × 10 | <10 | <10 | <3 |
| (ATCC 10231) | 0.5% aqueous solution | 4.1 × 10⁵ | 8.0 × 10 | <10 | <10 | <10 |
| | 1.0% aqueous solution | 4.2 × 10⁵ | 6.7 × 10 | <10 | <10 | <10 |
| | 1.5% aqueous solution | 4.3 × 10⁵ | 7.9 × 10 | <10 | <10 | <10 |
| | 2.0% aqueous solution | 4.8 × 10⁵ | 6.8 × 10 | <10 | <10 | <10 |
| | 4.0% aqueous solution | 5.9 × 10⁵ | 5.2 × 10 | <10 | <10 | <10 |
| | 4.0% sodium benzoate | 4.0 × 10⁵ | 3.4 × 10² | 3.0 × 10¹ | <10 | <10 |
| | (comparative) | | | | | |

The above comparison examples show that the method for preparing amphoteric glycolipid biosurfactant of the present invention is reasonable, and moreover, the synthetic biosurfactant has reached an integration of anions and cations. The described synthesis method of different strains at different stages is scientific, reasonable, and effective. The content of pure amphoteric glycolipid in the final fermentation stock solution reached an index above 45 g/L, which was higher than reported in the literature; and the compatibility of the surfactant was good.

The biosurfactants described herein can form ultra-low interfacial tension and surface tension with crude oil under different conditions, and has a wide ultra-low interfacial/surface tension.

The biosurfactants described herein have improved compatibility with divalent ions, such as calcium and magnesium.

The biosurfactants described herein have improved antibacterial properties.

According to particular embodiments of the present disclosure, the biosurfactant and amphoteric glycolipid biological surface active molecule preparations can be applied to industries such as petroleum, food, daily chemicals, and medicine.

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

It is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the example(s) or when application of the meaning renders any construction meaningless or essentially meaningless. In cases where the construction of the term would render it meaningless or essentially meaningless, the definition should be taken from Webster's Dictionary, 11th Edition or a dictionary known to those of ordinary skill in the art, such as the Oxford Dictionary of Biochemistry and Molecular Biology, 2^{nd} Edition (Ed. Anthony Smith, Oxford University Press, Oxford, 2006).

I claim:

1. An amphoteric glycolipid biosurfactant comprising:
an amphoteric glycolipid biological surface active molecule preparation produced by acid precipitation of fermentation culture supernatant produced by sequentially culturing Pseudomonas aeruginosa, Candida albicans, and Neurospora crassa in a fermentation medium comprising at least one hydrophilic carbon source and at least one hydrophobic carbon source;
one or more fatty acids;
one or more polysaccharides;
one or more sugar amines;
one or more lipopeptides;
ethanol;
one or more fatty amines; and
water,
wherein the amphoteric glycolipid biological surface active molecule preparation is the predominant active component.

2. The amphoteric glycolipid biosurfactant of claim 1, comprising:
0.5-3.0% fatty acids,
0.1-1.0% polysaccharide,
20.0-40.0% amphoteric glycolipid biological surface active molecule preparation,
0.1%-0.5% glycosamine,
0.01-0.2% lipopeptide,
1-3% ethanol, and
0.1-0.5% fatty amine.

3. The amphoteric glycolipid biosurfactant of claim 1, wherein the polysaccharide:
comprises a polysaccharide polymer comprising one or more of rhamnose, mannose, or glucose; and
has a molecular weight greater than 200,000 Daltons.

4. The amphoteric glycolipid biosurfactant of claim 1, wherein the fatty acid comprises a C₈-C₁₆ long-chain fatty acid.

5. The amphoteric glycolipid biosurfactant of claim 1, wherein the sugar amine comprises N-acetylglycosamine of glucose with a molecular weight of less than 1000 Daltons.

6. The amphoteric glycolipid biosurfactant of claim 1, wherein the lipopeptides comprise at least one lipopeptide containing 7-14 amino acid chains, with a molecular weight of 800-3000 Daltons.

7. The amphoteric glycolipid biosurfactant of claim 6, wherein the lipopeptides comprise one or more of subtilisin, a phenazine, and/or iturin.

8. The amphoteric glycolipid biosurfactant of claim 7, wherein the lipopeptide comprises subtilisin and phenazine in a ratio of subtilisin to phenazine of 10:1-12:1.

9. The amphoteric glycolipid biosurfactant of claim 1, wherein the structure of at least one amphoteric glycolipid biological surface active molecule in the preparation comprises fatty amine or Glycosamine or both; and the molecule has a weight of less than 1400 Daltons.

10. The amphoteric glycolipid biosurfactant of claim 1, wherein amphoteric glycolipid biological surface active molecule preparation comprises one or more of:
17-L-[(2′-O-β-D-glucopyranosyl-β-D-glucosyl)-O-]-octadecenoic acid amine-6′,6″ diacetate;
17-L-[(2′-O-β-D-glucopyranosamine-β-D-rhamnosyl)-O-]-octadecenoic acid-6′,6″, diacetate;
17-L-[(2′-O-β-D-glucopyranosylamino-β-D-rhamnosyl)-O-]-octadecylenoic acid-6′,6″ diacetate;
17-L-[(2′-O-β-D-glucopyranosylamino-β-D-rhamnosyl)-O-]-18 Enoic acid-6′, 6″ diacetate; or
17-L-[(2′-O-β-D-glucopyranosyl-β-D-glucosyl)-O-]-octadecene acid amine-6′, 6″ diacetate.

11. The amphoteric glycolipid biosurfactant of claim 1, wherein the fatty amine comprises one or more of lauryl amide, palmitic acid amide, stearamide, oleic acid amide, or stearyl oleamide.

12. An amphoteric glycolipid biological surface active molecule preparation produced by acid precipitation of fermentation culture supernatant produced by sequentially culturing Pseudomonas aeruginosa, Candida albicans, and Neurospora crassa in a fermentation medium.

13. The amphoteric glycolipid biological surface active molecule preparation of claim 12, which is obtained by sequential cultivation of Candida, Pseudomonas, and Neurospora in the fermentation medium to produce a fermentation broth mixture, separation of fermentation culture supernatant from the fermentation broth mixture, acid precipitation of material from the fermentation culture supernatant to produce a precipitate, and filtration of the precipitate.

14. The amphoteric glycolipid biological surface active molecule preparation of claim 12, wherein one or more of:
the Candida are cultured at 33-37° C.;
the Pseudomonas are cultured at 23-26° C.;
the Neurospora are cultured at 28-32° C.;
the fermentation medium comprises at least one hydrophilic carbon source and at least one hydrophobic carbon source;
the Candida are cultured for 24-36 hours;
the Pseudomonas are cultured for 80-100 hours; and/or
the Neurospora are cultured for 36-48 hours.

15. The amphoteric glycolipid biological surface active molecule preparation of claim 14, wherein the at least one hydrophilic carbon source comprises sucrose, glucose, and/or molasses; and or wherein the at least one hydrophobic carbon source comprises vegetable oil, animal oil, and/or a petroleum hydrocarbon compound.

16. The amphoteric glycolipid biological surface active molecule preparation of claim 12, production of which further comprises an extraction process comprising:
(1) separation, comprising lowering the fermentation broth to pH=8.0-10.0, centrifuging the pH adjusted broth at *10000 g to remove bacterial debris and residual oil, and harvesting a clear middle liquid layer;
(2) acid precipitation, comprising adjusting the middle clear liquid layer to pH=2-3, and refrigerating at 4-10° C. for 24 hours to produce a precipitate;
(3) filtration, comprising collecting the precipitate through a ceramic membrane filter at 4-10° C., dissolving/suspending the precipitate in an aqueous solution of pH>8, and filtering the suspended solution through a ceramic membrane filter to obtain an aqueous preparation of amphoteric glycolipid biosurface active molecule; and, optionally
(4) concentrating preparation in vacuo at 50° C.

17. The amphoteric glycolipid biological surface active molecule preparation of claim 12, wherein the Pseudomonas is cultured at 33-37° C. for 24-36 hours, and then connected to Candida for 80-100 hours at 23-26° C. Finally, insert the Neurospora bacteria and cultivate at 28-32° C. for 36-48 hours.

18. The amphoteric glycolipid biological surface active molecule preparation of claim 12, wherein the fermentation medium is at pH 6-7 and comprises: NaNO₃: 0.4-2.4%, FeCl₂: 0.002-0.006%, NaH₂PO₄: 0.25-1.5%, K₂HPO₄: 0.25-1.8%, MgSO₄.7H₂O: 0.005-0.015%, KCl: 0.05%-0.3%, choline chloride: 0.05%-0.3%, fatty amine: 0.5-1.0%, corn syrup: 0.05%-0.3%, hydrophobic carbon source: 3-5%, hydrophilic carbon source: 0.1%-0.5%, yeast extract powder: 0.001-0.1%, and trace elements: Zn, Mn, Ca.

19. The amphoteric glycolipid biological surface active molecule preparation of claim 12, which comprises at least one molecule having the structure:
17-L-[(2′-O-β-D-glucopyranosyl-β-D-glucosyl)-O-]-octadecenoic acid amine-6′,6″ diacetate;
17-L-[(2′-O-β-D-glucopyranosamine-β-D-rhamnosyl)-O-]-octadecenoic acid-6′,6″, diacetate;
17-L-[(2′-O-β-D-glucopyranosylamino-β-D-rhamnosyl)-O-]-octadecylenoic acid-6′,6″ diacetate;
17-L-[(2′-O-β-D-glucopyranosylamino-β-D-rhamnosyl)-O-]-18 Enoic acid-6′, 6″ diacetate; or
17-L-[(2′-O-β-D-glucopyranosyl-β-D-glucosyl)-O-]-octadecene acid amine-6′, 6″ diacetate.

20. The amphoteric glycolipid biological surface active molecule preparation of claim 12, which exhibits improved surface activity, improved bacteriostasis activity, improved emulsification activity, or a combination of two or more thereof.

21. A method of making an amphoteric glycolipid biological surface active molecule preparation, the method comprising: acid precipitation of fermentation culture supernatant produced by sequentially culturing Pseudomonas aeruginosa, Candida albicans, and Neurospora crassa in a fermentation medium.

22. The method of claim 21, comprising sequential cultivation of Candida, Pseudomonas, and Neurospora in the fermentation medium to produce a fermentation broth mixture, separation of fermentation culture supernatant from the fermentation broth mixture, acid precipitation of material from the fermentation culture supernatant to produce a precipitate, and filtration of the precipitate.

23. The method of claim 21, wherein one or more of:
the Candida are cultured at 33-37° C.;
the Pseudomonas are cultured at 23-26° C.;
the Neurospora are cultured at 28-32° C.;
the fermentation medium comprises at least one hydrophilic carbon source and at least one hydrophobic carbon source;
the Candida are cultured for 24-36 hours;
the Pseudomonas are cultured for 80-100 hours; and/or
the Neurospora are cultured for 36-48 hours.

24. The method of claim 23, wherein the at least one hydrophilic carbon source comprises sucrose, glucose, and/or molasses; and or wherein the at least one hydrophobic carbon source comprises vegetable oil, animal oil, and/or a petroleum hydrocarbon compound.

25. The method of claim 21, production of which further comprises an extraction process comprising:
(1) separation, comprising lowering the fermentation broth to pH=8.0-10.0, centrifuging the pH adjusted broth at *10000 g to remove bacterial debris and residual oil, and harvesting a clear middle liquid layer;
(2) acid precipitation, comprising adjusting the middle clear liquid layer to pH=2-3, and refrigerating at 4-10° C. for 24 hours to produce a precipitate;
(3) filtration, comprising collecting the precipitate through a ceramic membrane filter at 4-10° C., dissolving/suspending the precipitate in an aqueous solution of pH>8, and filtering the suspended solution through a ceramic membrane filter to obtain an aqueous preparation of amphoteric glycolipid biosurface active molecule; and, optionally
(4) concentrating preparation in vacuo at 50° C.

26. The method of claim 21, wherein the Pseudomonas is cultured at 33-37° C. for 24-36 hours, and then connected to Candida for 80-100 hours at 23-26° C. Finally, insert the Neurospora bacteria and cultivate at 28-32° C. for 36-48 hours.

27. The method of claim 21, wherein the fermentation medium is at pH 6-7 and comprises: NaNO₃: 0.4-2.4%, FeCl₂: 0.002-0.006%, NaH₂PO₄: 0.25-1.5%, K₂HPO₄: 0.25-1.8%, MgSO₄.7H₂O: 0.005-0.015%, KCl: 0.05%-0.3%, choline chloride: 0.05%-0.3%, fatty amine: 0.5-1.0%, corn syrup: 0.05%-0.3%, hydrophobic carbon source: 3-5%, hydrophilic carbon source: 0.1%-0.5%, yeast extract powder: 0.001-0.1%, and trace elements: Zn, Mn, Ca.

28. The method of claim 17, which comprises at least one molecule having the structure:
17-L-[(2′-O-β-D-glucopyranosyl-β-D-glucosyl)-O-]-octadecenoic acid amine-6′,6″ diacetate;
17-L-[(2′-O-β-D-glucopyranosamine-β-D-rhamnosyl)-O-]-octadecenoic acid-6′,6″, diacetate;
17-L-[(2′-O-β-D-glucopyranosylamino-β-D-rhamnosyl)-O-]-octadecylenoic acid-6′,6″ diacetate;
17-L-[(2′-O-β-D-glucopyranosylamino-β-D-rhamnosyl)-O-]-18 Enoic acid-6′, 6″ diacetate; or
17-L-[(2′-O-β-D-glucopyranosyl-β-D-glucosyl)-O-]-octadecene acid amine-6′, 6″ diacetate.
